(19) United States
(12) Patent Application Publication
(10) Pub. No.: US 2026/0102198 A1
(43) Pub. Date: Apr. 16, 2026

(54) CRYOABLATION NEEDLE TO LINE SET CONNECTOR

(71) Applicant: Boston Scientific Scimed, Inc., Maple Grove, MN (US)

(72) Inventors: Abigail Blanchette Teschendorf, Blaine, MN (US); Mark Edward DiLoreto, Chaska, MN (US); John L. Teschendorf, Plymouth, MN (US); Andrew Kevin Zachman, St. Michael, MN (US)

(21) Appl. No.: 19/336,770

(22) Filed: Sep. 23, 2025

Related U.S. Application Data
(60) Provisional application No. 63/707,357, filed on Oct. 15, 2024.

(51) Int. Cl. A61B 18/02; A61B 18/00

(52) U.S. Cl. CPC A61B 18/02; A61B 2018/00172; A61B 2018/00577; A61B 2018/0262

(57) ABSTRACT

Embodiments herein relate to cryoablation systems. In an embodiment a cryosurgery system includes a cryoablation probe and a line set. The system can include a probe connector configured to sealingly connect the line set to the cryoablation probe. The probe connector can have a main body having a main body distal end configured to sealingly connect to the cryoablation probe. The probe connector can have main body central portion configured to sealingly fit within the line set return tube. The probe connector can have a main body proximal end having a protrusion. The probe connector can have a coupling body configured to sealingly connect the line set supply tube. The coupling body is configured to fit around the main body proximal end such that the protrusion of the main body engages with the slot of the coupling body.

This application claims the benefit of U.S. Provisional Application No. 63/707,357, filed October 15, 2024, the content of which is herein incorporated by reference in its entirety.

### FIELD

Embodiments herein relate to cryoablation systems and more particularly to probe connectors for cryoablation systems.

### BACKGROUND

During cryosurgery, a surgeon may deploy one or more cryoprobes to ablate a target area of a patient anatomy by freezing and thawing the tissue. In one example, a cryoablation probe uses the Joule-Thompson effect to produce cooling or heating of the probe tip. In such cases, the expansion of a cryofluid in the cryoablation probe from a higher pressure to a lower pressure without heat exchange to the environment leads to cooling of the device tip. Heat transfer between the expanded cryofluid and the outer walls of the cryoprobe leads to formation of an ice ball in the tissue around the tip and consequent cryoablation of the tissue.

### SUMMARY

In a first aspect, a cryosurgery system includes a cryoablation probe, and a line set can include a line set supply tube and a line set return tube surrounding the line set supply tube. The cryosurgery system can include a probe connector configured to sealingly connect the line set to the cryoablation probe. The probe connector can include a main body having a main body distal end configured to sealingly connect to the cryoablation probe. The probe connector can include a main body central portion configured to sealingly fit within the line set return tube and defining one or more mating features configured to engage with the line set return tube. The probe connector can include a main body proximal end having a protrusion. The probe connector can include a coupling body configured to sealingly connect to a distal end of the line set supply tube, and having a slot. In various embodiments, the coupling body can be configured to fit around the main body proximal end such that the protrusion of the main body engages with the slot of the coupling body. In a second aspect, in addition to one or more of the preceding or following aspects, or in the alternative to some aspects, the main body distal end can include a probe insertion portion configured to fit into a handle of the cryoablation probe. In a third aspect, in addition to one or more of the preceding or following aspects, or in the alternative to some aspects, the main body distal end can include a shoulder configured to be joined to the handle of the cryoablation probe. In a fourth aspect, in addition to one or more of the preceding or following aspects, or in the alternative to some aspects, the main body can be configured to attach to a heat exchanger of the cryoablation probe. In a fifth aspect, in addition to one or more of the preceding or following aspects, or in the alternative to some aspects, a proximal end of the heat exchanger can be configured to be joined to an inner surface of the main body. In a sixth aspect, in addition to one or more of the preceding or following aspects, or in the alternative to some aspects, the coupling body can be overmolded onto the distal end of the line set supply tube. In a seventh aspect, in addition to one or more of the preceding or following aspects, or in the alternative to some aspects, the one or more mating features of the main body central portion include barbs configured to form a sealing interference fit with the line set return tube. In an eighth aspect, in addition to one or more of the preceding or following aspects, or in the alternative to some aspects, the probe connector can further include a gasket configured to form a seal between the main body and the coupling body. In a ninth aspect, in addition to one or more of the preceding or following aspects, or in the alternative to some aspects, the gasket can be positioned within the coupling body proximally to the slot. In a tenth aspect, in addition to one or more of the preceding or following aspects, or in the alternative to some aspects, the protrusion of the main body can be configured to snap into the slot of the coupling body. In an eleventh aspect, in addition to one or more of the preceding or following aspects, or in the alternative to some aspects, the coupling body includes a polymeric material. In a twelfth aspect, a probe connector can be included that is configured to sealingly connect a line set to a cryoablation probe. The probe connector can include a main body can be included having a main body distal end configured to sealingly connect to the cryoablation probe. The probe connector can include a main body central portion configured to fit within a return tube of the line set and defining one or more mating features configured to engage with the return tube. The probe connector can include a main body proximal end having a protrusion. The probe connector can include a coupling body configured to sealingly connect to a distal end of a supply tube of the line set, and having a slot. In various embodiments, the coupling body can be configured to fit around the main body proximal end such that the protrusion of the main body engages with the slot of the coupling body. In a thirteenth aspect, in addition to one or more of the preceding or following aspects, or in the alternative to some aspects, the main body distal end can include a probe insertion portion configured to fit into a handle of the cryoablation probe. In a fourteenth aspect, in addition to one or more of the preceding or following aspects, or in the alternative to some aspects, the main body distal end can include a shoulder configured to be joined to the handle of the cryoablation probe. In a fifteenth aspect, in addition to one or more of the preceding or following aspects, or in the alternative to some aspects, the coupling body can be overmolded onto the distal end of the supply tube. In a sixteenth aspect, in addition to one or more of the preceding or following aspects, or in the alternative to some aspects, the one or more mating features of the main body central portion include barbs configured to form an interference fit with the line set return tube. In a seventeenth aspect, in addition to one or more of the preceding or following aspects, or in the alternative to some aspects, can further include a gasket configured to form a seal between the main body and the coupling body. In an eighteenth aspect, in addition to one or more of the preceding or following aspects, or in the alternative to some aspects, the protrusion of the main body can be configured to snap into the slot of the coupling body. In a nineteenth aspect, in addition to one or more of the preceding or following aspects, or in the alternative to some aspects, the coupling body includes a polymeric material. In a twentieth aspect, a method of forming a probe connector configured to sealingly connect a line set to a cryoablation probe can be included. The method can include providing a main body of the probe connector. The method can include forming a coupling body assembly by overmolding the coupling body onto a distal end of a supply tube of the line set. The method can include engaging a protrusion of the main body with a slot of the coupling body to sealingly connect the main body with the coupling body assembly. The method can include inserting a return tube of the line set over the supply tube, inserting a proximal end of the main body into a distal end of the return tube of the line set, the main body defining one or more mating features configured to engage with the return tube. The method can include joining a distal end of the main body onto a handle of the cryoablation probe.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures(FIGS.), in which:

FIG. 1 is a schematic view of a cryosurgery system in accordance with various embodiments herein.

FIG. 2 is a perspective, schematic view of a probe connector in accordance with various embodiments herein.

FIG. 3 is a perspective, schematic view of a probe connector with the line set return tube removed in accordance with various embodiments herein.

FIG. 4 is an exploded view of the probe connector without the line set return tube, in accordance with various embodiments herein.

FIG. 5 is a cross-sectional view of the probe connector in accordance with various embodiments herein.

FIG. 6 is a perspective, schematic view of a main body of a probe connector in accordance with various embodiments herein.

FIG. 7 is a perspective, schematic view of a coupling body of a probe connector in accordance with various embodiments herein.

FIG. 8 is a perspective view of a cross-section of the probe connector in accordance with various embodiments herein.

FIG. 9 is a flowchart of a method of forming a probe connector configured to sealingly connect a line set to a cryoablation probe in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Cryoablation systems utilize the expansion of a working fluid to generate a cooling effect. The temperature at the tip of the cryoablation probe can reach cryogenic temperatures (~140 Kelvin) to form an ice ball and ablate a region in a patient’s anatomy. In some embodiments, it is desirable to utilize a cryoablation system having a closed-loop working fluid circuit in which the expanded working fluid is recirculated.

To maximize the efficiency of the cryoablation system and preserve the working fluid, the components are configured to minimize the amount of working fluid that escapes from the cryoablation system. The connection of a line set to the cryoablation probe can be susceptible to working fluid leaks. Accordingly, a probe connector can be provided to form a leak-tight seal between the line set and the cryoablation probe.

In various embodiments, a cryosurgery system can include a cryoablation probe. The cryosurgery system can include a line set having a line set supply tube and a line set return tube surrounding the line set supply tube. The cryosurgery system can include a probe connector configured to sealingly connect the line set to the cryoablation probe. The probe connector can include a main body having a main body distal end configured to sealingly connect to the cryoablation probe. The probe connector can include a main body central portion configured to sealingly fit within the line set return tube and defining one or more mating features configured to engage with the line set return tube. The probe connector can include a main body proximal end having a protrusion. The probe connector can include a coupling body configured to sealingly connect to a distal end of the line set supply tube. In various embodiments, the coupling body is configured to fit around the main body proximal end such that the protrusion of the main body engages with a slot of the coupling body.

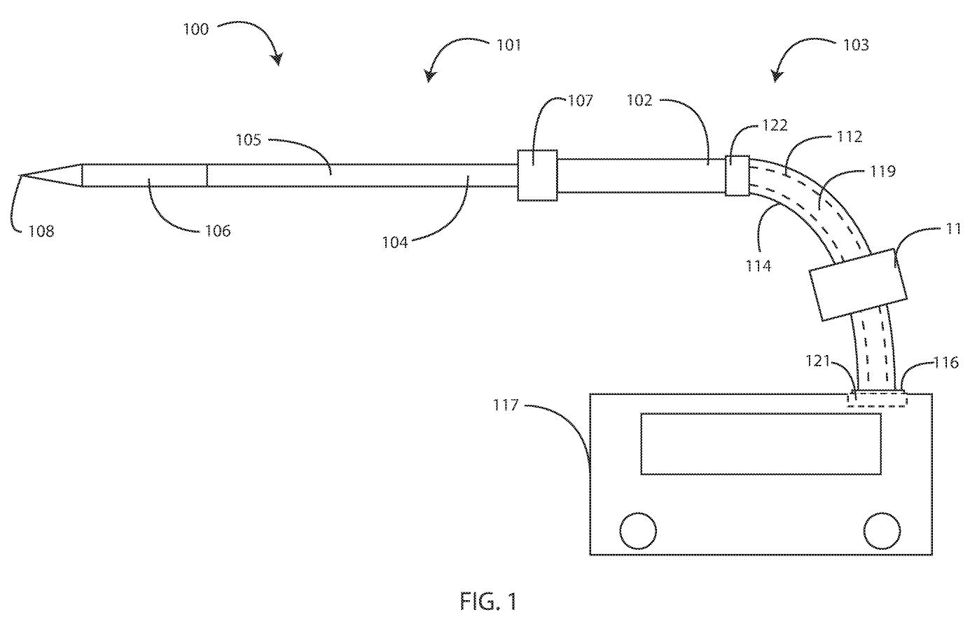

System Overview (FIG. 1)

Referring now to FIG. 1, a schematic view of a cryosurgery system 100 is shown in accordance with various embodiments herein. In various embodiments, the system can include a cryoablation probe 101 having a handle 102 and a shaft 104. In various embodiments, the shaft 104 is connected to the handle 102 with shaft-handle connector 107. In various embodiments, the shaft 104 and the shaft-handle connector 107 of a cryosurgery system 100 can form a catheter assembly.

In some embodiments, the cryosurgery system 100 includes a console 117. The console may be used to control the system and may be in electrical and fluid communication with the handle and cryoablation assembly. In various embodiments, the console 117 controls the flow of a working fluid. In the example of FIG. 1, the cryosurgery system 100 is a closed-loop system and the working fluid is contained within a closed-loop working fluid circuit within the cryoablation system.

In various embodiments the cryosurgery system 100 can include a cryoablation probe assembly 103. The cryoablation probe assembly 103 can include the cryoablation probe 101 and a line set 119. In various embodiments, line set 119 is configured to connect the console to the handle 102 of the cryoablation probe 101. In various embodiments, the line set 119 is configured to be flexible, enhancing the maneuverability of the cryoablation probe 101. In various embodiments, the line set 119 is formed from a polymeric material or materials including, but not limited to Acrylonitrile Butadiene Styrene (ABS), Nylon, polytetrafluoroethylene (PTFE), one or more polyether block amides (PEBA), or the like. In some embodiments, PEBA can include Pebax®, hereinafter “Pebax” and/or Vestamid®, hereinafter “Vestamid.” Pebax material is available from Arkema Inc., which has a business location at 900 First Avenue, King of Prussia, PA 19406, USA. Vestamid material is available from Evonik Inc., which has a business location at Essen, Rellinghauser Str. 1-11, Germany. In some embodiments, the line set 119 can be reinforced with a braided material, such as a stainless-steel braid, or the like.

In various embodiments, the cryoablation probe assembly 103 is configured to connect to a cryoablation probe port 121 of the console 117. In various embodiments, the cryosurgery system 100 can include a console connector 116 configured to sealingly connect the cryoablation probe assembly 103 to the cryoablation probe port 121 of the console 117. In various embodiments, the cryosurgery system 100 can further include a probe connector 122 configured to form a leak tight seal between the line set 119 and the handle 102 of the cryoablation probe 101.

In the example of FIG. 1, the working fluid is configured to circulate from the console 117 to the cryoablation probe 101 via a line set supply tube 112 and travel back from the cryoablation probe 101 to the console via a line set return tube 114. In the example of FIG. 1, the line set return tube 114 shares a common longitudinal axis with the line set supply tube 112. However, other configurations are possible such as the line set supply tube 112 being arranged adjacent to the line set return tube 114.

In various embodiments, the working fluid circuit runs through both the handle 102 and the shaft 104 of the cryosurgery system 100 and carries the fluid which generates the ice ball. The term “fluid circuit” is used throughout the application, and could be replaced with gas circuit, liquid circuit, fluid chamber, gas chamber, or liquid chamber in various embodiments. The term “fluid” is used throughout and could be replaced with gas or liquid in various embodiments. The working fluid is circulated through the probe to generate an ice ball in the patient’s body surrounding an expansion chamber 106. The expansion chamber 106 defines the portion of the shaft 104 that is not insulated to increase thermal conduction and where the ice ball is generated. In various embodiments, the shaft 104 carries high pressure working fluid from the handle 102 to the expansion chamber 106, where it undergoes a Joule-Thompson effect and correspondingly experiences a temperature change. The working fluid exits down the shaft 104, through the handle 102, and continues to circulate through the closed-loop working fluid circuit. In various embodiments, the shaft 104 may include an insulated zone 105. The insulated zone 105 defines the portion of shaft 104 that is insulated to reduce thermal losses and define the shape of the ice ball.

The working fluid can be a cooling fluid or mixture of fluids. In some embodiments, the pressure of the high-pressure stream of the working fluid flowing from the handle 102 to the expansion chamber 106 can be greater than or equal to 2.0 MPa, 2.3 MPa, 2.7 MPa, or 3.0 MPa. In some embodiments, the pressure of the high-pressure stream of the working fluid can be less than or equal to 12.0 MPa, 9.0 MPa, 6.0 MPa, or 3.0 MPa. In some embodiments, the pressure of the high-pressure stream of the working fluid can fall within a range of 2.0 MPa to 12.0 MPa, or 2.3 MPa to 9.0 MPa, or 2.7 MPa to 6.0 MPa, or can be about 3.0 MPa. Pressure measurements are provided as absolute pressure measurements herein.

Accordingly, the temperature of the working fluid at the expansion chamber 106 can be about 140 Kelvin. In some embodiments, the temperature of the working fluid at the expansion chamber 106 can be greater than or equal to 100 Kelvin, 113 Kelvin, 127 Kelvin, or 140 Kelvin. In some embodiments, the temperature of the working fluid at the expansion chamber 106 can be less than or equal to 200 Kelvin, 180 Kelvin, 160 Kelvin, or 140 Kelvin. In some embodiments, the temperature of the working fluid at the expansion chamber 106 can fall within a range of 100 Kelvin to 200 Kelvin, or 113 Kelvin to 180 Kelvin, or 127 Kelvin to 160 Kelvin, or can be about 140 Kelvin.

In various embodiments, the cryosurgery system 100 can further include a heat exchanger 118. The heat exchanger 118 can be positioned in any suitable position within the cryosurgery system 100. In the embodiment of FIG. 1, the heat exchanger 118 is disposed in a hub surrounding a line set 119 connecting the cryoablation probe 101 to the console 117. In various embodiments, the heat exchanger 118 is configured to cool the working fluid prior to the working fluid reaching the expansion chamber 106 of the cryoablation probe 101. The heat exchanger 118 can be any suitable heat exchanger such as a recuperative heat exchanger, an electric cooler, or the like.

In some embodiments, the outer surface of the shaft 104 may be thermally insulated from the inner surface of the shaft. In various embodiments, shaft insulation can be provided by a sealed vacuum chamber or containing a non-circulating fluid or gas within a sealed chamber. In alternative embodiments, a vacuum circuit runs through both the handle 102 and the insulated zone 105 of the shaft 104. Vacuum is actively pulled along the insulated zone 105 of the shaft 104 throughout the cryoablation procedure, providing a protective barrier between the outer surface of the shaft 104 and the patient. In alternative embodiments, shaft insulation can be obtained by circulating fluid, gas, or a heated fluid throughout the shaft or by electrically heating portions of the shaft.

The distal end of the shaft 104 may terminate in a distal operating tip 108. During use, the distal operating tip 108 is deployed in the body of a patient, is surrounded by tissue, and cryogenically ablates the tissue in some instances. The distal operating tip 108 may be advantageously configured to pierce tissue in some instances. For example, the distal operating tip 108 may include a sharp tip, such as a trocar tip. Alternatively, the distal operating tip 108 may not be a sharp tip. In some embodiments, the distal operating tip 108 can be an atraumatic tip designed to cause minimal tissue injury. In some embodiments, the distal operating tip 108 may also contain a working port configured for any of aspiration, delivery of therapeutics, and delivery of other devices including, but not limited to guide wires, imaging catheters, sensing devices, biopsy devices, balloons, and stents.

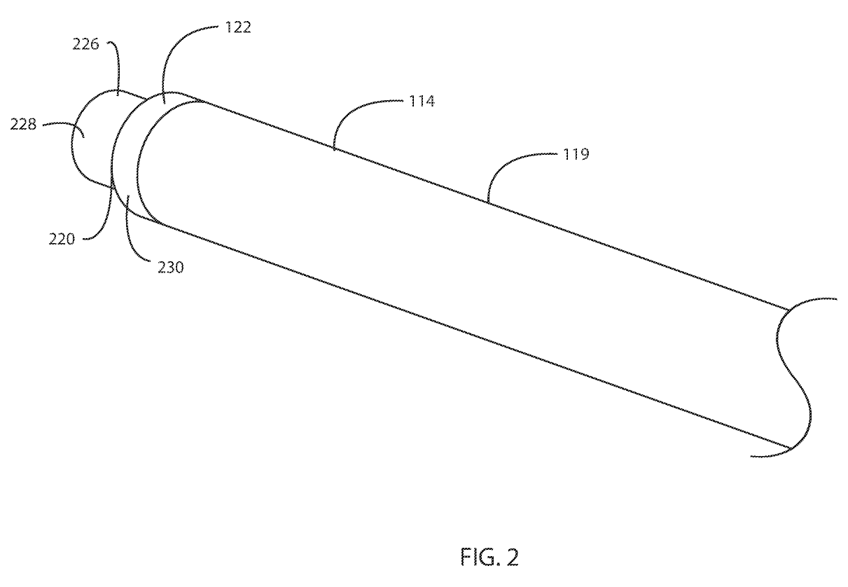

Probe Connector (FIGS. 2-5)

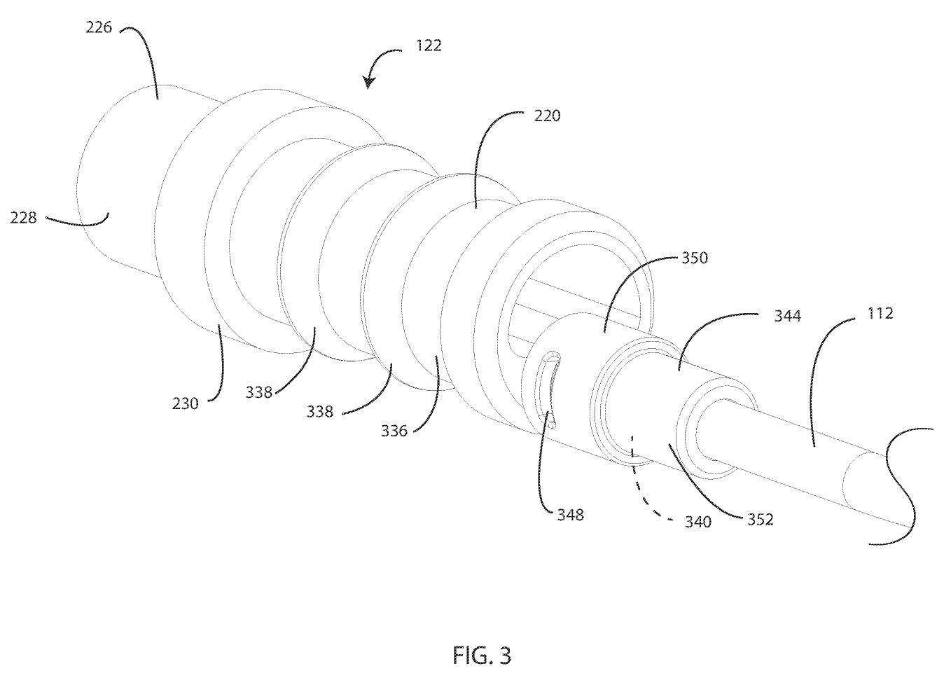

Referring now to FIGS. 2-3, various views of a probe connector connected to a line set of a cryoablation probe assembly are shown in accordance with various embodiments herein. FIG. 2 depicts a perspective, schematic view of a probe connector in accordance with various embodiments herein. FIG. 3 depicts a perspective, schematic view of a probe connector with the line set return tube removed in accordance with various embodiments herein.

In various embodiments, the line set 119 includes a supply tube 112 surrounded by a return tube 114. In various embodiments, the probe connector 122 is configured to sealingly connect a cryoablation probe 101 (FIG. 1) to a distal end of the line set 119. By sealingly connect, it is meant that the probe connector 122 is configured to connect the cryoablation probe 101 to the line set 119 such that substantially none of the circulating working fluid escapes from the cryosurgery system 100.

In various embodiments, the probe connector 122 can include a main body 220. In various embodiments, the main body 220 can be formed from any suitable material or materials such as Nitinol (NiTi), stainless steel, or the like. The main body 220 can include a main body distal end 226 configured to sealingly connect to the cryoablation probe 101.

In various embodiments, the main body distal end 226 can include a probe insertion portion 228. In various embodiments, the probe insertion portion 228 is configured to fit into a handle 102 of the cryoablation probe 101. In various embodiments, the handle 102 of the of the cryoablation probe 101 defines a cavity at its proximal end having an inner diameter. In various embodiments, the outer diameter of the probe insertion portion 228 is approximately equal to the inner diameter of handle cavity. In such an embodiment, the probe insertion portion 228 is configured to fit tightly into the handle cavity of the cryoablation probe 101.

In various embodiments, the main body distal end 226 can include a shoulder 230 configured to be joined to the handle 102 of the cryoablation probe 101. In various embodiments, the outer diameter of the shoulder 230 is larger than the inner diameter of the handle cavity. In various embodiments, the main body distal end 226 is configured to be inserted into the handle 102 of the cryoablation probe 101 such that probe insertion portion 228 is inside of the handle cavity and the shoulder 230 abuts the proximal end of the handle 102. In various embodiments, the shoulder 230 can be joined to the handle 102 of the cryoablation probe 101 by any suitable process such as welding, or the like. In an embodiment, the shoulder 230 of the main body 220 is welded to the handle 102 with a full rotation weld to seal the probe connector 122 to the cryoablation probe 101 and prevent leaks of the working fluid to the environment.

In various embodiments, the probe connector 122 is configured to be inserted into distal end of the return tube 114. As best seen in FIG. 2 the probe connector 122 is inserted into distal end of the return tube 114 such that the distalmost end of the return tube abuts the shoulder 230. In various embodiments, the outer diameter of the return tube 114 is approximately equal to the outer diameter of the shoulder 230 creating a seamless interface between the return tube and the shoulder. In various embodiments, the distal end of the return tube 114 abuts the opposite side of the shoulder 230 to the proximal end of the handle 102.

Referring to FIG. 3, in various embodiments, the main body 220 includes a main body central portion 336. In various embodiments, the main body central portion 336 is configured to sealingly fit within the line set return tube 114 when the probe connector 122 is inserted into the line set 119. In various embodiments, the main body central portion 336 can define one or more mating features 338 configured to engage with the line set return tube 114. In the example of FIG. 3, the main body central portion 336 defines a plurality of barbed mating features 338 configured to form an interference fit with the with the line set return tube 114.

Alternatively, the main body central portion 336 can include any suitable number or configuration of mating features 338 that are configured to form a seal with the return tube 114 of the line set 119 and prevent the working fluid from leaking from the interface between the return tube 114 and the probe connector 122. Additional or alterative mating features for sealing the return tube 114 to the probe connector 122 can include any of flanges, crimp bands, heat shrink coverings, or the like.

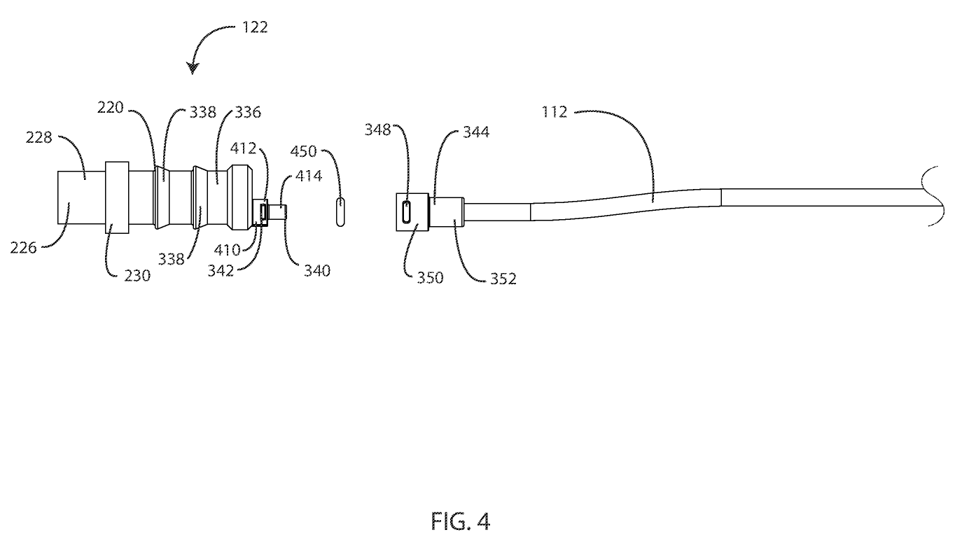

In various embodiments, the probe connector 122 can include a main body proximal end 340. The main body proximal end 340 can further include a protrusion 342 (visible in FIG. 4). In the view of FIG. 3, the protrusion 342 has not yet been snapped into place within a slot 348, so the protrusion 342 is not shown within the slot 348 in FIG. 3.

In various embodiments, the probe connector 122 can include a coupling body 344 configured to engage with the main body 220 at the main body proximal end 340. In various embodiments, the coupling body 344 can be formed from a polymeric material or materials, including, but not limited to polyimide, fluorinated ethylene propylene (FEP), Teflon, polytetrafluoroethylene (PTFE), or the like. In some embodiments, the coupling body 344 can be formed from a resin, such as a Pebax based resin, an ABS based resin, a nylon-based resin, Vestamid-based resin, or the like. In some embodiments, the resin can contain glass-filled fibers that are configured to reduce shrinking and expansion of the coupling body 344 caused by temperature fluctuations during a cryoablation procedure. In various embodiments, the coupling body 344 is configured to sealingly connect to a distal end of the supply tube 112 of the line set 119.

In some embodiments, the coupling body 344 is overmolded onto the distal end of the supply tube 112 of the line set 119. Overmolding, as defined herein refers to molding a first component (e.g., the coupling body 344) over or around a second component (e.g., the supply tube 112) using any suitable manufacturing process such as injection molding, or the like. In various embodiments, overmolding the coupling body 344 to the supply tube 112 provides a leak-tight seal and prevents the working fluid from leaking from the interface between the line set 119 and the probe connector 122.

In various embodiments, the main body proximal end 340 is configured to fit within the coupling body 344. An outer diameter of the main body proximal end 340 can be approximately equal to an inner diameter of the coupling body 344 such that the main body proximal end 340 is configured to fit tightly within the coupling body.

In various embodiments, the coupling body 344 can include a slot 348. In various embodiments, the coupling body 344 is configured to fit around the main body proximal end 340 such that the protrusion 342 of the main body 220 engages with the slot 348 of the coupling body 344. For instance, the protrusion 342 of the main body 220 can be configured to snap into the slot 348 of the coupling body 344. In various embodiments, the protrusion 342 includes a ramped surface and is configured to have some flexibility to facilitate insertion into the coupling body 344.

In various embodiments, the configuration of the probe connector 122 results in a device that separates the supply flow of the working fluid from the return flow of the working fluid and prevents any of the working fluid to the environment. Moreover, the probe connector 122 is configured to tightly retain each component in place to prevent any torque and damage to the system components due to twisting of the device during use.

Referring now to FIG. 4, an exploded view of the probe connector is shown in accordance with various embodiments herein. In various embodiments, the probe connector 122 can include a main body 220 that is configured to fit within the coupling body 344.

In various embodiments, the main body 220 includes a nose portion 410 extending proximally from the remainder of the main body 220 and is received within the coupling body 344. In various embodiments, the nose portion 410 includes a base nose portion 412 and a tip nose portion 414, where the tip nose portion 414 extends proximally from the base nose portion 412.

The base nose portion 412 includes a protrusion 342 and the coupling body 344 can include a slot 348. In various embodiments, the coupling body 344 is configured to fit around the nose portion 410 such that the protrusion 342 of the main body 220 engages with the slot 348 of the coupling body 344. The slot 348 of the coupling body 344 can be sized and shaped to securely hold the protrusion 342. For instance, the protrusion 342 is sized to fit into the slot with a small enough clearance to limit the relative motion between the main body 220 and the coupling body 344.

In an alternative configuration, the main body 220 can include a slot and the coupling body 344 can include a protrusion configured to snap into the slot. Alternatively, the main body 220 and the coupling body 344 can have any other suitable set of mating features to couple the main body to the coupling body and limit the relative motion between the main body 220 and the coupling body 344.

In various embodiments, the nose portion 410 of the main body 220 is off-center compared to the remainder of the main body 220, where the nose portion 410 includes a longitudinal axis that is parallel with and spaced apart from a main body longitudinal axis. In alternative embodiments, the nose portion 410 of the main body 220 can be centered compared to the remainder of the main body 220. The base nose portion 412 and tip nose portion 414 can include a cylindrical body and a cylindrical outer surface, where the protrusion 342 extends from the cylindrical outer surface of the base nose portion 412. The base nose portion 412 has a base outer diameter measured at a location that does not overlap with the protrusion 342 and the tip nose portion 414 has a tip outer diameter, where the base outer diameter is larger than the tip outer diameter.

In various embodiments, the probe connector 122 can further include a gasket 450. The gasket 450 can be configured to form a seal between the main body 220 and the coupling body 344. In various embodiments, the gasket is disposed around the tip nose portion 414 and trapped between the remainder of the main body 220 and the coupling body 344 when the main body is attached to the coupling body.

Referring to FIG. 3, the coupling body 344 includes a base portion 350 and a neck portion 352 extending proximally from the base portion 350. The base portion 350 has a first outer diameter and the neck portion 352 has a second outer diameter that is smaller than the first outer diameter. The base portion 350 has a first inner diameter and the neck portion 352 has a second inner diameter that is smaller than the first outer diameter.

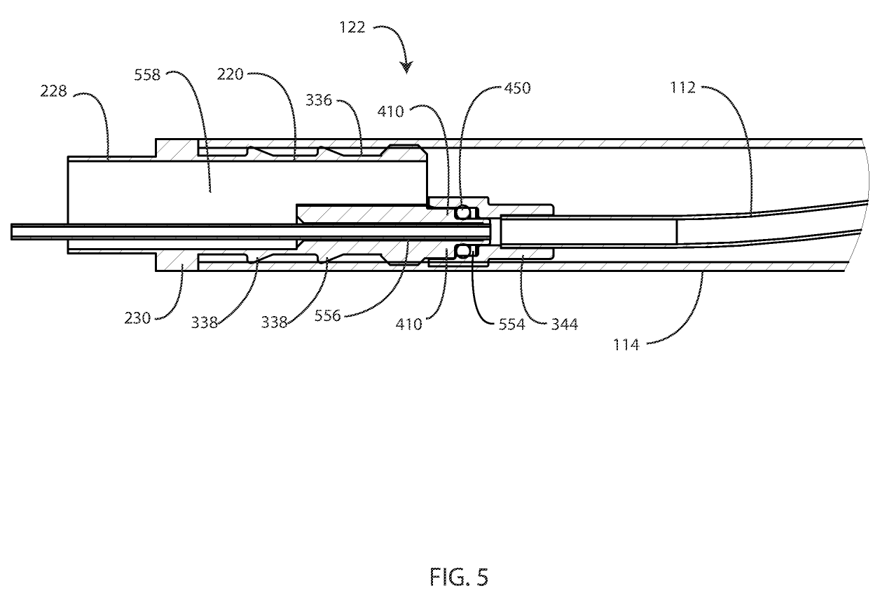

Referring now to FIG. 5, a cross-sectional view of the probe connector is shown in accordance with various embodiments herein. In various embodiments, the main body 220 of the probe connector 122 includes a nose portion 410 that is configured to fit within a cavity 554 of the coupling body 344. The probe connector 122 is configured to be inserted into a distal end of the return tube 114 such that the distalmost end of the return tube abuts the shoulder 230 of the main body 220.

In various embodiments, the main body central portion 336 is configured to sealingly fit within the line set return tube 114 when the probe connector 122 is inserted into the return tube such that mating features 338 of the main body 220 engage with the line set return tube. In the example of FIG. 5, the barbed mating features 338 of the main body are configured to form an interference fit with an inner surface of the return tube 114, creating a leak tight seal between the probe connector 122 and the return tube and holding the line set 119 in position with respect to the probe connector. In some embodiments, one or more crimp bands can be wrapped around the probe connector 122 and the return tube 114 to further enhance the leak tight seal between the probe connector and the return tube.

In various embodiments gasket 450 is included to enhance the seal between the main body 220 and the coupling body 344. In the example of FIG. 5, the gasket 450 can be positioned within the cavity 554 of the coupling body 344 proximally to the slot 348. Alternatively, the gasket 450 can be positioned around the main body 220 distally to the protrusion 342. After positioning the gasket 450, the main body can be inserted into the cavity 554 of the coupling body 344, compressing the gasket 450 and forming a seal between the main body and the coupling body.

In various embodiments, the main body 220 can further include a probe engagement portion 556. In various embodiments, a portion of the cryoablation probe 101 is configured to be inserted into the distal end of the main body 220 such that the probe is held in place by the probe engagement portion 556. In some embodiments, the portion of the cryoablation probe can be joined to the probe engagement portion 556 by any suitable means such as welding, or the like.

In various embodiments, a supply tube of the cryoablation probe 101 is configured to be in fluid communication with the line set supply tube 112 such that working fluid supplied at the console 117 can flow to the cryoablation probe 101 via the line set supply tube.

In various embodiments, the main body 220 can define a return flow lumen 558. In various embodiments, the return flow lumen 558 is configured to be in fluid communication with both the line set return tube 114 and a return tube of the cryoablation probe (not shown in this view) such that working fluid returning from the expansion chamber 106 of the cryoablation probe 101 can return to the console 117 via the line set return tube.

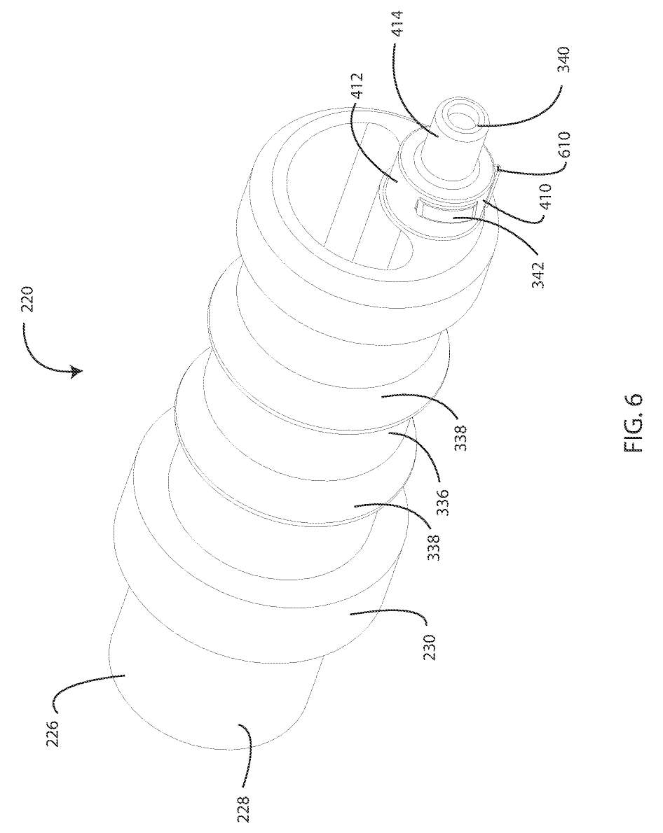

### Main Body (FIG. 6)

Referring now to FIG. 6, a perspective, schematic view of a main body 220 of a probe connector is shown in accordance with various embodiments herein. In various embodiments, the main body 220 can include a main body distal end 226 having a probe insertion portion 228 that is configured to be inserted into the handle 102 of a cryoablation probe 101. The main body 220 can further include a main body central portion 336 having one or more mating features 338 configured to engage with the line set return tube 114. The main body 220 can further include a main body proximal end 340 and a nose portion 410 having a protrusion 342. The nose portion 410 includes a base nose portion 412 and a tip nose portion 414, where the tip nose portion 414 extends proximally from the base nose portion 412. In various embodiments, the base nose portion 412 further comprises an optional alignment feature 610 extending away from a longitudinal axis of the nose portion 410. The alignment feature 610 can be a ridge, peak, bulge or other structure. The alignment feature 610 facilitates the appropriate orientation of the main body 220 when the nose portion 410 is inserted into the coupling body 344.

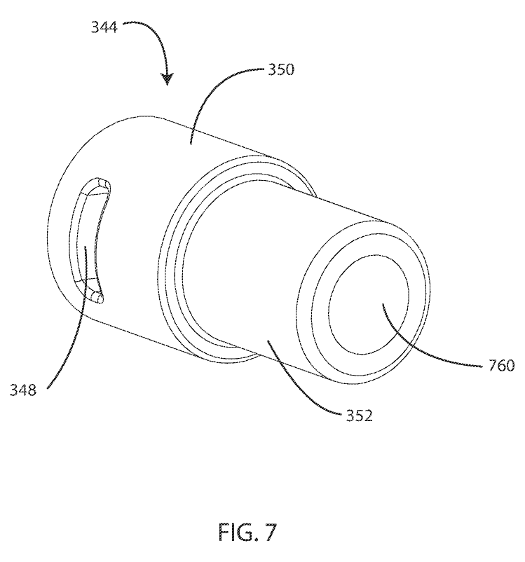

### Coupling Body (FIG. 7)

Referring now to FIG. 7, a perspective, schematic view of a coupling body 344 of a probe connector is shown in accordance with various embodiments herein. In various embodiments, an inner surface 760 of the coupling body 344 is configured to be overmolded onto the distal end of the supply tube 112 of the line set 119. In various embodiments, the coupling body 344 can include a slot 348. The coupling body 344 is configured to fit around the main body proximal end 340 such that the protrusion 342 of the main body 220 engages with the slot 348 of the coupling body 344.

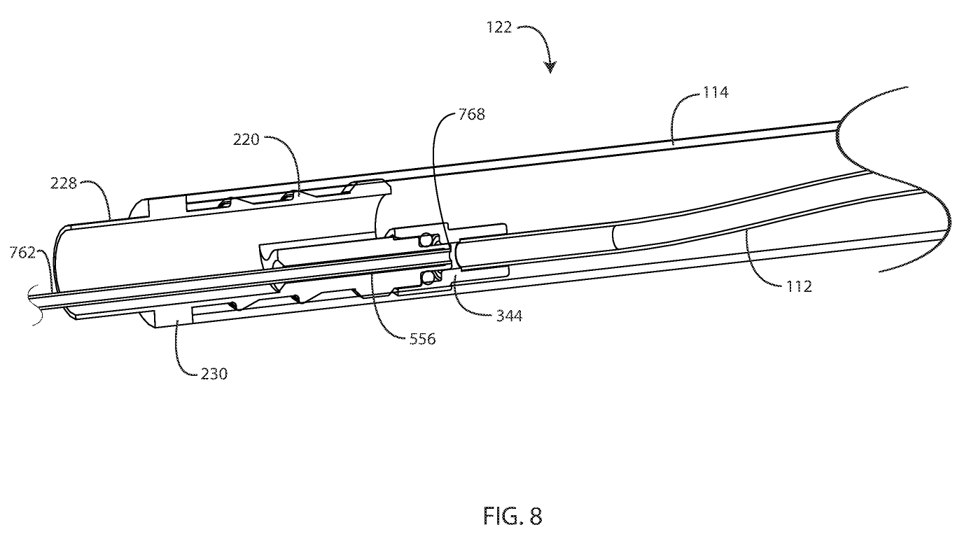

### Heat Exchanger Configuration (FIG. 8)

Referring now to FIG. 8, a perspective view of a cross-section of a probe connector 122 is shown in accordance with various embodiments herein. In various embodiments, the probe connector 122 can include a main body 220 that is configured to fit within the coupling body 344. The probe connector 122 is configured to be inserted into distal end of the return tube 114 such that the distalmost end of the return tube abuts the shoulder 230 of the main body 220.

In various embodiments, the distal end of the probe connector 122 is configured to connect to the cryoablation probe 101. The main body 220 of the probe connector 122 can include a probe insertion portion 228 that is configured to fit into a handle 102 of the cryoablation probe 101 and a probe engagement portion 556 configured to connect to a heat exchanger 762 of the cryoablation probe.

In various embodiments, the probe connector 122 is further configured to attach to a heat exchanger 762 of the cryoablation probe 101. In various embodiments, a proximal end of the heat exchanger 762 is configured to be joined to an inner surface of the main body 220. In the example of FIG. 8, the proximal end of the heat exchanger 762 is configured to be joined to the main body 220 along the probe engagement portion 556. The proximal end of the heat exchanger 762 is configured to be joined to the main body by any suitable means, such as welding, or the like. For instance, the proximal end of the heat exchanger 762 can be welded to the main body 220 using a full rotation weld configured to prevent the heat exchanger from twisting during use.

In an embodiment, the outer diameter of the proximal end of the heat exchanger 762 is configured to fit tightly within the probe engagement portion 556 of the main body 220. Upon inserting the heat exchanger 762 into the probe engagement portion 556, a full rotation weld can be formed at the interface 768 between the heat exchanger and the main body.

In various embodiments, the working fluid is configured to flow from the line set supply tube 112 into the proximal end of the heat exchanger at interface 768. In such embodiments, the weld between the heat exchanger 762 and the main body 220 is further configured to form a leak tight seal and to prevent the working fluid from leaking at the interface between the supply tube and the heat exchanger.

In various embodiments, the heat exchanger 762 can include a proximal portion. In the example of FIG. 8, the proximal portion of the heat exchanger 762 is tube shaped. In various embodiments, the proximal portion of the heat exchanger 762 can extend from the probe connector 122 into the handle 102 of the cryoablation probe 101.

The heat exchanger 762 can further include a distal portion. The distal portion of the heat exchanger can be positioned within the handle 102 and/or a proximal portion of the shaft 104 of the cryoablation probe 101. In various embodiments, the distal portion of the heat exchanger can be operably connected to the working fluid circuit and is configured to precool the working fluid from the working fluid circuit prior to the working fluid being delivered to the expansion chamber 106 of the cryoablation probe 101. In some embodiments, the distal end of the heat exchanger 762 can take the form of a helical tube wound around a central core.

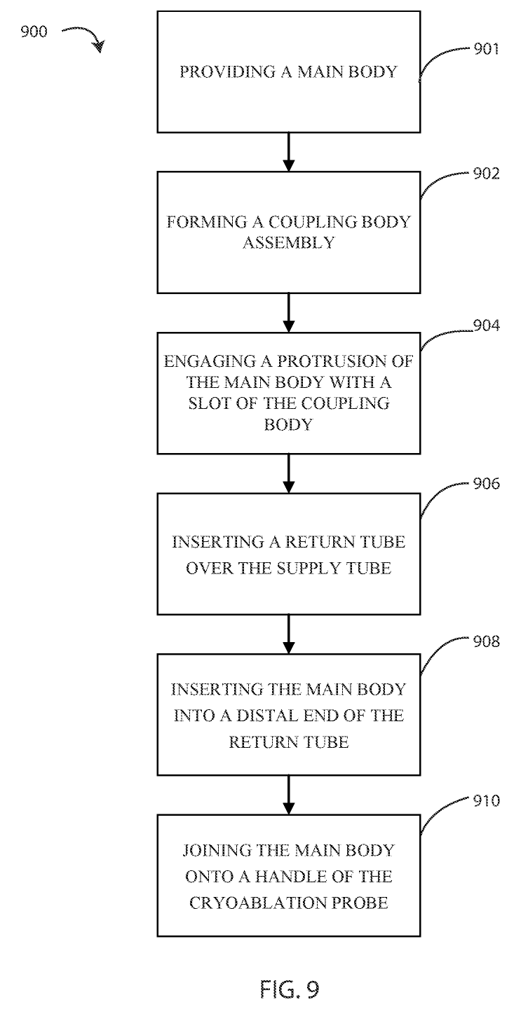

Method of Forming a Probe Connector (FIG. 9)

Many different methods are contemplated herein, including, but not limited to, methods of making, methods of using, and the like. Aspects of system/device operation described elsewhere herein can be performed as operations of one or more methods in accordance with various embodiments herein. Referring now to FIG. 9, a method 900 of forming a probe connector configured to sealingly connect a line set to a cryoablation probe is described in accordance with various embodiments herein. In various embodiments, the method 900 can include the step 901 of providing a main body 220 of the probe connector 122.

In various embodiments, the method 900 can include the step 902 of forming a coupling body assembly. In some various embodiments, step 902 can include overmolding the coupling body 344 onto a distal end of a supply tube 112 of the line set 119. In various embodiments, overmolding the coupling body 344 to the supply tube 112 provides a leak-tight seal and prevents the working fluid from leaking from the interface between the line set 119 and the probe connector 122.

In various embodiments, the method 900 can include the step 904 of engaging a protrusion 342 of the main body 220 with a slot 348 of the coupling body 344 to sealingly connect the main body 220 with the coupling body assembly. In some embodiments, step 904 includes engaging a proximal end of the main body 220 with a cavity 554 the coupling body 344.

In various embodiments, the method 900 can include the step 906 of inserting a return tube 114 of the line set 119 over the supply tube 112. In various embodiments, the coupling body 344 has been overmolded the distal end of the supply tube 112 and the step 906 can include pulling a distal end of the return tube 114 up to the coupling body 344.

In various embodiments, the method 900 can include the step 908 of inserting a proximal end of the main body 220 into a distal end of the return tube 114 of the line set 119. In some embodiments, step 908 includes engaging one or more mating features 338 of the main body 220 with an inner surface of the return tube 114.

In various embodiments, the method 900 can include the step 910 of joining a distal end of the main body 220 onto a handle 102 of the cryoablation probe 101. In some embodiments, step 910 includes inserting a probe insertion portion 228 of the main body 220 into the handle of the cryoablation probe 101. In some embodiments, step 910 includes joining a shoulder 230 of the main body 220 to the handle of the cryoablation probe, but any suitable means, such as welding or the like.

It should be noted that, as used in this specification and the appended claims, the singular forms "a," "an," and "the" include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase "configured" can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cryosurgery system, comprising:
a cryoablation probe; and
a line set comprising a line set supply tube and a line set return tube surrounding the line set supply tube;
a probe connector configured to sealingly connect the line set to the cryoablation probe, the probe connector comprising:
a main body comprising:
a main body distal end configured to sealingly connect to the cryoablation probe;
a main body central portion configured to sealingly fit within the line set return tube and defining one or more mating features configured to engage with the line set return tube;
a main body proximal end comprising a protrusion;
a coupling body configured to sealingly connect to a distal end of the line set supply tube, and comprising a slot;
wherein the coupling body is configured to fit around the main body proximal end such that the protrusion of the main body engages with the slot of the coupling body.

2. The cryosurgery system of claim 1, the main body distal end comprising a probe insertion portion configured to fit into a handle of the cryoablation probe.

3. The cryosurgery system of claim 2, the main body distal end comprising a shoulder configured to be joined to the handle of the cryoablation probe.

4. The cryosurgery system of claim 1, wherein the main body is configured to attach to a heat exchanger of the cryoablation probe.

5. The cryosurgery system of claim 4, wherein a proximal end of the heat exchanger is configured to be joined to an inner surface of the main body.

6. The cryosurgery system of claim 1, wherein the coupling body is overmolded onto the distal end of the line set supply tube.

7. The cryosurgery system of claim 1, wherein the one or more mating features of the main body central portion comprise barbs configured to form a sealing interference fit with the line set return tube.

8. The cryosurgery system of claim 1, the probe connector further comprising a gasket configured to form a seal between the main body and the coupling body.

9. The cryosurgery system of claim 8, wherein the gasket is positioned within the coupling body proximally to the slot.

10. The cryosurgery system of claim 1, wherein the protrusion of the main body is configured to snap into the slot of the coupling body.

11. The cryosurgery system of claim 1, wherein the coupling body comprises a polymeric material.

12. A probe connector configured to sealingly connect a line set to a cryoablation probe, the probe connector comprising:
a main body comprising:
a main body distal end configured to sealingly connect to the cryoablation probe;
a main body central portion configured to fit within a return tube of the line set and defining one or more mating features configured to engage with the return tube;
a main body proximal end comprising a protrusion;
a coupling body configured to sealingly connect to a distal end of a supply tube of the line set, and comprising a slot;
wherein the coupling body is configured to fit around the main body proximal end such that the protrusion of the main body engages with the slot of the coupling body.

13. The probe connector of claim 12, the main body distal end comprising a probe insertion portion configured to fit into a handle of the cryoablation probe.

14. The probe connector of claim 13, the main body distal end comprising a shoulder configured to be joined to the handle of the cryoablation probe.

15. The probe connector of claim 12, wherein the coupling body is overmolded onto the distal end of the supply tube.

16. The probe connector of claim 12, wherein the one or more mating features of the main body central portion comprise barbs configured to form an interference fit with the line set return tube.

17. The probe connector of claim 12, further comprising a gasket configured to form a seal between the main body and the coupling body.

18. The probe connector of claim 12, wherein the protrusion of the main body is configured to snap into the slot of the coupling body.

19. The probe connector of claim 12, wherein the coupling body comprises a polymeric material.

20. A method of forming a probe connector configured to sealingly connect a line set to a cryoablation probe comprising:
providing a main body of the probe connector;
forming a coupling body assembly by overmolding the coupling body onto a distal end of a supply tube of the line set; and
engaging a protrusion of the main body with a slot of the coupling body to sealingly connect the main body with the coupling body assembly;
inserting a return tube of the line set over the supply tube;
inserting a proximal end of the main body into a distal end of the return tube of the line set, the main body defining one or more mating features configured to engage with the return tube; and
joining a distal end of the main body onto a handle of the cryoablation probe.
